(19) United States
(12) Patent Application Publication
(10) Pub. No.: US 2005/0080140 A1
(43) Pub. Date: Apr. 14, 2005

(54) Method for treating or preventing inflammatory disorders

(75) Inventors: Toshihisa Hatae, Osaka-shi (JP); Chieko Yokoyama, Suita-shi (JP); Tadashi Tanabe, Toyonaka-shi (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: AnGes MG, Inc., Toyonaka-shi (JP)

(21) Appl. No.: 10/923,348

(22) Filed: Aug. 20, 2004

Related U.S. Application Data
(60) Provisional application No. 60/511,003, filed on Oct. 13, 2003.

(30) Foreign Application Priority Data
Feb. 25, 2004 (JP) 2004-049299

(51) Int. Cl. A61K 31/557

(52) U.S. Cl. 514/573

(57) ABSTRACT

According to the present invention, the gene for IL-13, an anti-inflammatory cytokine, was shown to be a target of prostacyclin (PGI₂)-activated Peroxisome proliferator-activated receptor (PPAR) δ. Furthermore, the PGI₂-PPARδ signaling pathway was revealed to regulate the expression of IL-13 gene in human vascular endothelial cells, and controls inflammatory responses induced by proinflammatory cytokines through the production of IL-13 in an autocrine or paracrine manner. Thus, the present invention provides a method for treating or preventing inflammatory disorders, which comprises administering a therapeutically effective amount of PPARδ agonist into a subject. Furthermore, the present invention provides a method for treating or preventing inflammatory disorders, which comprises administering a prostacyclin synthase gene or a protein encoded by the gene into a subject.

The present application is related to U.S. Ser. No. 60/511,003, filed on Oct. 13, 2003, which is incorporated herein by reference.

### FIELD OF THE INVENTION

The present invention relates to a method for treating or preventing inflammatory disorders through the activation of peroxisome proliferator-activated receptor (PPAR) δ. Specifically, the invention provides a method for treating or preventing inflammatory disorders wherein a therapeutically effective amount of a PPARδ agonist, or a prostacyclin synthase gene or a protein encoded by the gene is administered to a subject.

### BACKGROUND OF THE INVENTION

Peroxisome proliferator-activated receptors (PPARs) are members of the nuclear-hormone-receptor superfamily of ligand-dependent transcription factors, and regulate gene expression by binding to PPAR-response elements (PPREs) in the regulatory regions of their target genes (Kersten et al., Nature (2000) 405: 421-4; Lazar, Nat. Med. (2001) 7: 23-4). Three mammalian PPAR subtypes, termed PPARα, PPARγ and PPARδ, have been identified (Kersten et al., Nature (2000) 405: 421-4). Many of the function of PPARα and PPARγ are associated with inflammation, tumorigenesis, pathways of lipid metabolism and atherosclerosis (Kersten et al., Nature (2000) 405: 421-4; Lazar, Nat. Med. (2001) 7: 23-4). PPARδ has been proposed to be a mediator of fatty acid oxidation, energy dissipation, skin wound healing, tumorigenesis and cell fate control (Wang et al., Cell (2003) 113: 159-70; Berger and Moller, Annu. Rev. Med. (2002) 53: 409-35; Hatae et al., J. Biol. Chem. (2001) 276: 46260-7). However, unlike PPARα and PPARγ, little is known about the functions of PPARδ in the cardiovascular system.

Prostaglandins (PGs) that play roles in a wide spectrum of clinical and biological processes are known to activate ligands for PPARs (Kersten et al., Nature (2000) 405: 421-4; Lazar, Nat. Med. (2001) 7: 23-4; Hatae et al. , J. Biol. Chem. (2001) 276: 46260-7). PGs are a family of lipid mediators derived from arachidonic acid (AA) (Samuelsson et al., Annu. Rev. Biochem. (1978) 47: 997-1029; Smith et al., Annu. Rev. Biochem. (2000) 69: 145-82; Moncada and Vane, N. Engl. J. Med. (1979) 17: 1142-7; Vane and Botting, Am. J. Cardiol. (1995) 75: 3A-10A; Tanabe and Ullrich, J. Lipid Medial. Cell Signal. (1995) 12: 243-55; Tanabe and Tohnai, Prostaglandins Other Lipid Mediat. (2002) 68-69: 95-114). AA is converted to an intermediate, prostaglandin H₂ (PGH₂) by two isoforms of cyclooxygenase, COX-1 and COX-2. COX-2 has important roles in inflammation, and the gene for COX-2 is up-regulated by proinflammatory cytokines, such as tumor necrosis factor (TNF)-α and interleukin (IL)-1β.

The COX product PGH₂ is converted to one of several prostanoids, including prostaglandin D₂ (PGD) and prostacyclin (PGI₂), by specific synthases (Hatae et al., J. Biol. Chem. (2001) 276: 46260-7; Samuelsson et al., Annu. Rev. Biochem. (1978) 47: 997-1029; Vane and Botting, Am. J. Cardiol. (1995) 75: 3A-10A). It is known that PGD metabolite 15-deoxy-Δ^{12,14}-prostaglandin J₂ (15d-PGJ₂) is an activator of PPARγ (Kersten et al., Nature (2000) 405: 421-4; Forman et al., Cell (1995) 83: 803-12). Nevertheless, the physiological relevance of 15d-PGJ₂ remains unclear due to indefinite certification of the existence of 15d-PGJ₂ in vivo.

On the other hand, recent studies have demonstrated that endogenous PGI₂ is a natural ligand for PPARδ (Hatae et al., J. Biol. Chem. (2001) 276: 46260-7; Lim and Dey, Endoclinology (2002) 143: 3207-10). PGI₂, a potent vasodilator and endogenous inhibitor of platelet aggregation, is a clinically important factor that influences the well-being of endothelium and its function (Vane and Botting, Am. J. Cardiol. (1995) 75: 3A-10A; Wu and Thiagarajan, Annu. Rev. Med. (1996) 47: 315-31; Luscher et al., Annu. Rev. Med. (1993) 44: 395-418; Wall and Harker, Annu. Rev. Med. (1980) 31: 361-71).

The endothelium, a continuous cellular monolayer lining the blood vessels, has an enormous range of important homeostatic roles, and disturbance of these homeostatic balances by endogenous or exogenous factors induces disease states characterized by inflammation and atherosclerosis (Cines et al., Blood (1998) 91: 3527-61; Luscinskas and Gimbrone, Annu. Rev. Med. (1996) 47: 413-21). Therefore, abnormal PGI₂-signaling is considered to have important roles in impaired vascular responses in inflammatory and atherosclerotic vessels (Wu and Thiagarajan, Annu. Rev. Med. (1996) 47: 315-31; Cines et al., Blood (1998) 91: 3527-61). Although PGI₂ has been classically considered to function through a G protein-coupled receptor (IP) linked to Gs, increasing cAMP (Hatae et al., J. Biol. Chem. (2001) 276: 46260-7; Samuelsson et al., Annu. Rev. Biochem. (1978) 47: 997-1029), little is known regarding other cellular pathway(s) of PGI₂. The findings in the previous study of the present inventors indicate that the activation of PPARδ by PGI₂ plays important roles in PGI₂-dependent regulation of the cell fate of vascular endothelial cells (Hatae et al., J. Biol. Chem. (2001) 276: 46260-7). However, the molecular details of PGI₂-PPARδ signal transduction system remains to be clarified.

The vascular endothelial layer participates in the maintenance of homeostasis and a wide variety of pathophysiological processes in the cardiovascular system (Fishman, Ann N.Y. Acad. Sci. (1982) 401: 1-8). Endothelial cells are also the site of action of many endogenous hormones, including PGI₂, exogenous drugs and toxic substances, all of which induce various functional changes in the vascular endothelium, leading to alterations in gene expression and cellular dysfunction (Cines et al., Blood (1998) 91: 3527-61; Luscinskas and Gimbrone, Annu. Rev. Med. (1996) 47: 413-21) Endothelial dysfunction, resulting in chronic inflammation of the vascular wall, proliferation of smooth muscle cells and formation of foam cells, contributes to the development of atherosclerosis (Wu and Thiagarajan, Annu. Rev. Med. (1996) 47: 315-31; Cines et al., Blood (1998) 91: 3527-61; Luscinskas and Gimbrone, Annu. Rev. Med. (1996) 47: 413-21).

The PPARs, receptors for pathophysiological and pharmacological agonists, are expressed in vascular tissues including endothelial cells (Kersten et al., Nature (2000) 405: 421-4; Lazar, Nat. Med. (2001) 7: 23-4). Recent studies revealed important roles for PPARα and PPARγ in the regulation of inflammatory responses, foam-cell formation and plaque stability (Duval et al., Trends Mol. Med. (2002) 8: 422-30; Neve et al., Biochem. Pharmacol. (2000) 60: 1245-50; Plutzky, Am. J. Cardiol. (2001) 88: 10K-15K) Therefore, PPARs are potential targets for the treatment of vascular inflammatory disease and atherosclerosis. However, little is known about the relationship between PPARδ and vascular homeostasis.

### SUMMARY OF THE INVENTION

The present invention provides a method for treating or preventing inflammatory disorders, which comprises administering prostacyclin proliferator-activated receptor (PPAR) δ agonist into a subject. Such PPARδ agonists include prostacyclin analogs that are penetrable into mammalian cells, such as prostacyclin (epoprostenol), beraprost, taprostene, nileprost and OP-2507; carbaprostacyclin analogs, such as carbaprostacyclin, iloprost, cicaprost, ciprostene, treprostinil and bonsentan; and pharmaceutically acceptable salts thereof.

Furthermore, another aspect of the present invention is a method for treating or preventing inflammatory disorder by administering a prostacyclin synthase gene or a protein encoded by the gene into a subject.

According to the present invention, interleukin-13 (IL-13) mediated ameliorable inflammatory disorders, such as rheumatoid arthritis, rheumatoid spondylitis, osteoarthritis, gouty arthritis, inflammatory bowel disease, Crohn's disease, Guillain-Barre syndrome, schleroderma, fibrosis, dermatitis, psoriasis, angioedema, eczematous dermatitis, hyperproliferative skin disease, inflammatory skin conditions, glomerulonephritis, nephritis, vascular inflammation, atherosclerosis, angitis, phlebitis, arteritis, aorititis, post PTCA restenosis, post by-pass surgery restenosis, transplantation rejection, anaphylaxis, sepsis, thrombosis, ischemia/reperfusion injury and autoimmune diseases are treated. Transplantation rejection that can be treated according to the present method includes renal allograft rejection, cardiac allograft rejection and transplantation-associated vasculopathy.

### BRIEF DESCRIPTION OF THE DRAWINGS

FIG. 1 depicts the effects of exogenous cPGI on the production of IL-13 in primary cultured human vascular endothelial cells. FIG. 1A shows the result of reverse transcription-polymerase chain reaction (RT-PCR). Human vascular endothelial cells (1.2×10⁴ cells/cm²) were treated with cPGI (10 μM) for 12 h and the expression of IL-13 mRNA (upper panel) and β-actin as an internal control (lower panel) was detected. FIG. 1B shows the result of ELISA. Human vascular endothelial cells (1.2×10⁴ cells/cm²) were treated with cPGI (0, 1, 10 and 100 μM) for 24 h and amounts of IL-13 protein expressed into the culture media were measured. FIG. 1C shows the result of reporter assay. Human vascular endothelial cells (1.2×10⁴ cells/cm²) were transfected with a luciferase vector that contained three copies of PPRE (PPREx3-Luc) and cultured in EGM-2 containing 2% FBS with cPGI (0, 1, 10 and 100 μM). After 24 h, luciferase activities were measured. Results represent means±S.D. of three experiments.

FIG. 2 depicts the existence of five copies of PPRE consensus element in the promoter sequence of the human IL-13 gene (SEQ ID NO: 22). FIG. 2A shows a part of the promoter sequence of the human IL-13 gene comprising five copies of PPRE consensus element from −407 to −288. FIG. 2B shows the result of reporter assay. Human vascular endothelial cells (1.2×10⁴ cells/cm²) were transfected with luciferase (Luc) reporter vector containing 1 bp, 377 bp, 413 bp or 286 bp of the promoter region of the IL-13 gene and treated with (+) or without (−) cPGI (10 μM). After 24 h, luciferase activities were measured. The region containing putative PPRE consensus sequences was shown to be essential for PGI₂-dependent expression of IL-13 in primary cultured human vascular endothelial cells. Results represent means±S.D. of three experiments.

FIG. 3 depicts the effects of dominant negative forms of PPARδ (PPARδdn) and CREB (CREBdn) on cPGI-dependent activation of human IL-13 gene. Human vascular endothelial cells (1.2×10⁴ cells/cm²) were cotransfected with IL-13-luciferase reporter vector containing the 413 bp fragment of the IL-13 promoter and expression vector for wild-type PPARδ (PPARδwt), dominant negative PPARδ (PPARδdn), wild-type CREB (CREBwt) or dominant negative CREB (CREBdn), and treated with cPGI (10 μM). After 24 h, luciferase activities were measured. Results represent means±S.D. of three experiments.

FIG. 4 depicts the effects of TNF-α, exogenous cPGI and anti-human IL-13 monoclonal antibody on the adhesion between primary cultured human vascular endothelial cells and human monocytic leukocyte U937 cells. FIG. 4A shows the effect of TNF-α. Monolayers of human vascular endothelial cells (1.2×10⁴ cells/cm²) were incubated with TNF-α (0, 0.1, 1 or 10 ng/ml) at 37° C. for 24 h, and PGI₂ production from the cells was measured as the amount of 6-keto-PGI_{1α}. FIG. 4B shows the effect of TNF-α and cPGI. Monolayers of human vascular endothelial cells (1.2×10⁴ cells/cm²) were transfected with a luciferase reporter vector containing three copies of PPRE (PPREx3-Luc) and cultured at 37° C. for 24 h. After treatment of the cells with TNF-α (0, 0.1, 1 or 10 ng/ml) and cPGI (0, 2 or 10 μM) for 24 h, luciferase activities of the reporter vector were measured. FIG. 4C shows the influence of TNF-α and cPGI on IL-13 production. Monolayers of human vascular endothelial cells (1.2×10⁴ cells/cm²) were incubated with TNF-α (0, 0.1, 1 or 10 ng/ml) and cPGI (0, 2 or 10 μM) at 37° C. for 24 h, and the production of IL-13 from the cells was measured. FIG. 4D shows the result of adherence assay. Confluent monolayers of human vascular endothelial cells in 12-well plates were preincubated with TNF-α (0 or 10 ng/ml) and anti-IL-13 monoclonal antibody (0 or 0.5 μg/ml), cPGI (0 or 10 μM) or recombinant human IL-13 (0 or 10 ng/ml) at 37° C. for 24 h. Calcein-labeled monocytic leukocyte U937 cells (8×10⁴ cells/well) were added to the pretreated endothelial cells and adherence assays were performed. Results represent means±S.D. of three experiments. FIG. 4E shows the result of the adherence assay over time. Confluent monolayers of human vascular endothelial cells in 12-well plates were preincubated with TNF-α (0 or 10 ng/ml) and cPGI (0 or 10 μM) with or without anti-IL-13 monoclonal antibody (0.5 μg/ml) at 37° C. for 24 h. Human monocytic leukocyte U937 cells (8×10⁴ cells/well in 12-well plates) were labeled with 2.5 μM calcein-AM at room temperature for 45 min, added to the pretreated endothelial cells and incubated with cPGI (0 or 10 μM) and anti-IL-13 monoclonal antibody (0 or 0.5 μg/ml) for 30 min at room temperature to perform the adherence assays. Results represent means of three experiments.

FIG. 5 depicts the effects of exogenous cPGI and anti-human IL-13 monoclonal antibody on COX-2 expression induced by a combination of TNF-α and IL-1β in primary cultured human vascular endothelial cells. Human vascular endothelial cells (1.2×10⁴ cells/cm²) were pretreated with TNF-α (1 ng/ml) and IL-1β (20 ng/ml) for 1 h, followed by cPGI (0 or 10 μM), anti-human IL-13 monoclonal antibody (0 or 0.5 μg/ml) or control IgG (0 or 0.5 μg/ml). After 12 h, changes of COX-2 gene expression in the cells were analyzed by RT-PCR.

FIG. 6 depicts the effect of endogenous production of PGI₂ and exogenous cPGI on the regulation of endogenous IL-13 expression in primary cultured human vascular endothelial cells. FIG. 6A shows the result of Western analysis. A mixture of four siRNAs specific for human PGIS or non-silencing control was transfected into primary cultured human vascular endothelial cells. Lanes 1 and 2: control; and lane 3: siRNA mixture-treated cells. The treatment of the cells with the siRNAs for 10 days decreased the endogenous expression of PGIS. FIG. 6B shows the effect of siRNA and TNF-α detected by ELISA. Human vascular endothelial cells treated with siRNA mixture or non-silencing control were cultures in EGM-2 containing 2% FBS and TNF-α (0 or 10 ng/ml) for 24 h. The amount of PGI₂ produced into the culture medium was measured as the amount of 6-keto-PGF_{1α}. The production of 6-keto-PGF_{1α} was completely suppressed in siRNA-treated cells. N.D. indicates not detectable. Results represent means±S.D. of three experiments. FIG. 6C shows the effects of siRNA treatment and exogenous addition of cPGI on endogenous production of IL-13 in human vascular endothelial cells. Cells treated with siRNA or control non-silencing RNAi were cultured in EGM-2 containing 2% FBS, TNF-α (0 or 10 ng/ml) and CPGI (0 or 10 μM) for 24 h, and amounts of IL-13 expressed into the culture medium were measured by ELISA. Results represent means±S.D. of three experiments.

FIG. 7 depicts the nucleotide sequence (SEQ ID NO: 1) of the prostacyclin synthase gene and the amino acid sequence (SEQ ID NO: 2) of the synthase encoded by the gene.

### DETAILED DESCRIPTION OF THE INVENTION

The words “a”, “an” and “the” as used herein mean “at least one” unless otherwise specifically indicated.

The aim of the present study was to determine the target genes and molecular mechanism of the PGI₂-PPARδ signaling pathway. Microarray analysis and dominant negative forms of PPARδ and cAMP-responsive element binding protein (CREB) were used to identify a target that responds to PPARδ activation with PGI₂. In addition, primary cultured human vascular endothelial cells were used to investigate the relationship between the regulation of the target gene via the PGI₂-PPARδ pathway and events in the control of vascular inflammation or the early process of atherosclerosis, such as monocyte-endothelial cell adhesion and COX-2 gene expression induced by proinflammation cytokines in human vascular endothelium.

In this study, for the first time, IL-13 gene was shown to be an important target of PPARδ activated with PGI₂. The promoter of the human IL-13 gene has a short region containing five copies of a PPRE consensus element that is essential for PGI₂-PPARδ signaling-dependent expression of IL-13. Although cAMP-mediated phosphorylation of transcription factor GATA-3 has been reported to regulate IL-13 gene (Hatae et al., FEBS Lett. (1996) 389: 268-72), the present experiment demonstrated that coexpression of dominant negative mutant PPARδ suppressed IL-13 gene expression in human vascular endothelial cells. Furthermore, coexpression of dominant negative CREB had little or no effect on PGI₂-mediated activation of the human IL-13 gene. These data suggest that PPARδ is a key regulator of PGI₂-dependent expression of the human IL-13 gene in human vascular endothelial cells.

According to the present invention, it was also shown that PPARδ-mediated production of IL-13 from vascular endothelial cells significantly suppresses TNF-α-induced adhesion between monocytes and endothelial cells. Proinflammatory cytokines such as TNF-α and IL-1 play a critical role in the control of endothelial cell function, modulating many cellular responses (Tracey and Cerami, Annu. Rev. Med. (1994) 45:491-503; Gamble et al., Proc. Natl. Acad. Sci. U.S.A. (1989) 86: 7169-73). Adherence of human monocytes to vascular surfaces is an essential step in the pathological process of inflammation and atheroma (Wasserman et al., Am. J. Cardiol. (2003) 91: 34A-40A). Indeed, TNF-α has been detected in atherosclerotic plaques with immunoreactive TNF-α in atheroscleoric but not in normal arteries (Barath et al., Am. J. Cardiol. (1990) 65: 297-302). Under conditions of inflammation, vascular endothelial cells are gradually stimulated by TNF-α released by damaged cells to become proadhesive and procoagulant (Libby et al., J. Cardiovasc. Pharmacol. (1995) 25(suppl.2): S9-12). It is known that stimulation of endothelial cells by TNF-α increases the surface expression of leukocyte adhesion molecules (Frangogiannis et al., Cardiovac. Res. (2002) 53: 31-47). These responses of endothelial cells to TNF-α, including expression of adhesion molecules for mononuclear cells, are important in the pathogenesis of atherosclerosis (Paleolog et al., Blood (1994) 84: 2578-90). It has also been reported that human vascular endothelial cells become adhesive for leukocytes through increased cell surface expression of E-selectin after activation by TNF-α, and that immunohistochemical analysis of human atheroscelerotic arteries shows increased expression of E-selectin in areas of intimal thickening and atheromatous plaques (van der Wal et al., Am. J. Pathol. (1992) 141:1427-33). In the presence of TNF-α, endothelial cells show increased E-selectin expression and adhesion of U937 cells (Paysant et al., Endothelium (2002) 9: 263-71). IL-13 has been reported to suppress both the expression of E-selectin mRNA in and adhesion of leukocytes to TNF-α-activated or IL-1-activated endothelial cells (Etter et al., Cytokine (1998) 10: 395-403). These studies also strongly support the findings demonstrated herein.

In addition, PGI₂/PPARδ-mediated production of IL-13 was found to abrogate the expression of COX-2 induced by proinflammatory cytokines. Several IL-13-triggered events are potentially involved in the inhibition of COX-2 expression. Another report that supports the result of the present invention, for example, IL-13 has been shown to induce the expression of IL-1 decoy receptor (Colotta et al., J. Biol. Chem. (1994) 269: 12403-6), and to promote the expression of an IL-1 receptor antagonist (Muzio et. al., Blood (1994) 83: 1738-43). These effects would reduce the continued activation of COX-2 by autocrine or paracrine mechanisms. Thus, the PGI₂-PPARδ-IL-13 pathway may have an important role in the regulation of COX-2 gene expression and control of inflammation in vascular endothelial cells.

Moreover, RNA-interference (RNAi) methods revealed that endogenous PGI₂ production contributes to the regulation of endogenous IL-13 expression in response to the stimulation of TNF-α in human vascular endothelial cells. In addition, exogenous addition of carbaprostacyclin (cPGI) significantly induced endogenous expression of IL-13 in human vascular endothelial cells depleted of PGI₂ by treatment with siRNA specific for PGI₂ synthase (PGIS). Therefore, cPGI may also have a beneficial effect on the maintenance of blood vessel homeostasis in PGI₂-deprived cells such as senescent vascular endothelium.

In summary, a novel PGI₂-PPARδ signaling pathway was revealed to regulate expression of the target IL-13 gene, stimulates endogenous expression of IL-13 protein, inhibits monocyte-endothelium adhesion and COX-2 expression induced by proinflammatory cytokines in an autocrine or paracrine manner. Therefore, the PGI₂-PPARδ-IL-13 pathway in human vascular cells may provide a potential target for the treatment of vascular inflammatory diseases. The present results suggest new roles for the PGI₂-PPARδ signaling pathway in inflammatory responses in vascular endothelial cells. Furthermore, together with the results from the previous studies of the inventors, it may explain the beneficial effect of PGI₂ on diseases including vascular inflammation and atheroscleosis.

Thus, the present invention provides a method for treating or preventing inflammatory disorders, which comprises administering a therapeutically effective amount of PPAR δ agonist into a subject.

According to the invention, the treatment or prevention of the disease is achieved by administering a PPARδ agonist into the subject. Herein, the phrase “PPARδ agonist” refers to substances that, upon binding to the receptor, changes the conformation of the receptor and induces physiological functions. Substances can be tested for their PPARδ agonist activity by contacting the substance with cells expressing PPARδ, detecting their binding with PPARδ and then detecting signals that serve as the indicator of the activation of PPARδ. Such signals include IL-13 expression level, which can be detected as in the Example. The substance should preferably be penetrable into mammalian cells. Such substances include prostacyclin analogs, such as prostacyclin (epoprostenol), beraprost, taprostene, nileprost and OP-2507; carbaprostacyclin analogs, such as carbaprostacyclin, iloprost, cicaprost, ciprostene, treprostinil and bonsentan; and pharmaceutically acceptable salts thereof; but the present invention is not restricted thereto.

In the present invention, the PPARδ agonist is administered alone or formulated as a preparation. The agonist may be administered orally, parenterally, subcutaneously, interdermally, intravascularly, etc. Moreover, for example, the agonist may be solubilized in a sterile pharmaceutically acceptable solution, such as distilled water, phosphate buffered saline (PBS), etc., as needed. In addition, the solution may comprise buffers, colorants, corrigents, diluents, disintegrators, emulsifiers, excipients, extenders, fragrances, preservatives, solubilizers, stabilizers, sweetners, etc. Using such additives, the agonist maybe formulated into capsules, elixirs, emulsions, granules, injections, pills, powders, solutions, suspensions, syrups, tablets, troches, dragees, gels, etc. as needed.

The dose of the PPARδ agonist depends on the kind of selected agonist, administration route, and the weight, age, symptom, sex and such of the subject to be treated; but those skilled in the art can determine a preferable dose by considering such conditions.

Furthermore, RNAi-based silencing of the PGIS gene in human vascular endothelial cells was revealed to decrease IL-13 production and exogenous addition of cPGI increased endogenous IL-13 expression in such cells. Namely, endogenous production of PGI₂ was shown to contribute to IL-13 expression in the cells. Therefore, it is expected that increased endogenous PGIS levels in human vascular endothelial cells enhance the production of PGI2 and secretion of IL-13. Thus, the present invention relates to a method for treating or preventing inflammatory disorders by enhancing the in vivo level of PGIS in cells. The in vivo level of PGIS in cells can be achieved, for example, by administering a prostacyclin synthase gene. Thus, the present invention provides a method for treating or preventing inflammatory disorders by administering a prostacylcin synthase gene into a subject and expressing the gene in vivo.

Herein, the phrase “prostacyclin synthase gene” refers to any kind of polynucleotide that encodes a protein that catalyzes the reaction of prostaglandin endoperoxide to PGI₂ homologues. Such polynucleotides may be composed of DNA, RNA or a combination thereof. Furthermore, the polynucleotide may comprise other synthetic nucleic acids so long as it is expressed in vivo upon introduction into a subject. The polynucleotide may be the whole genomic prostacyclin synthase gene or the whole cDNA encoding the prostacyclin synthase, or even parts thereof so long as it is translated into a protein that has the biological function of the prostacyclin synthase.

The human prostacyclin synthase gene having the sequence of SEQ ID NO: 1 can be mentioned as a preferable example. Furthermore, the gene of the present invention may encode a mutant protein derived from nature. Such natural mutant proteins include allelic mutants, alternative isoforms, etc. However, the present invention is not restricted thereto and includes polynucleotides that encode a protein with equivalent function to the human prostacyclin synthase.

The phrase “functionally equivalent” herein means that the protein has the biological activity of a prostacyclin synthase, i.e., catalyzes the reaction of prostaglandin endoperoxide to PGI₂ homologues. Thus, the “prostacyclin synthase gene” used in the present invention includes: (1) polynucleotides consisting of the nucleotide sequence from the 28th base to the 1527th base of SEQ ID NO: 1; (2) polynucleotides encoding the amino acid sequence of SEQ ID NO: 2; (3) polynucleotides hybridizing under stringent conditions to the nucleotide of (1); (4) polynucleotides encoding a protein having a homology of about 70% or more to the amino acid sequence of SEQ ID NO: 2; and (5) polynucleotides encoding the amino acid sequence of SEQ ID NO: 2, wherein one or more amino acids are added, deleted, inserted and/or substituted. All of these polynucleotides are encompassed in the prostacyclin synthase gene to be used in the present invention, so long as the protein encoded by the polynucleotides retain the function of the prostacyclin synthase gene.

The polynucleotides encoding the amino acid sequence of SEQ ID NO: 2 include polynucleotides consisting of the nucleotide sequence from the 28th base to the 1527th base of SEQ ID NO: 1 as well as those with other nucleotide sequence due to the degeneracy of the genetic code.

Using the known prostacyclin synthase gene (e.g., SEQ ID NO: 1) or a portion thereof as a probe or primer, a polynucleotide hybridizing thereto may be obtained. Such polynucleotides. hybridizing to the prostacyclin synthase gene is expected to encode a protein that has equivalent function to the prostacyclin synthase and thus are included in the prostacyclin synthase gene used for the present invention. Conditions for hybridization to obtain polynucleotides encoding a protein with equivalent function to the prostacyclin synthase generally may include a wash condition of “1×SSC, 37° C. It is known that a polynucleotide with higher homology to the probe sequence can be obtained by employing a condition with higher stringency, such as a wash condition of “0.5×SSC, 0.1% SDS, 42° C.” or “0.1×SSC, 0.1% SDS, 65° C.”. However, these conditions are merely examples, and it should be understood that those skilled in the art can select a suitable condition for hybridization, taking the nucleotide sequence, concentration of the reagent, concentration and length of the probe, reaction temperature, reaction time and such into consideration.

Proteins encoded by polynucleotides that can be isolated according to such hybridization or PCR methods generally show a high homology to known prostacyclin synthases at both their nucleotide sequence level and amino acid sequence level, and encode a protein with similar functions to that encoded by the polynucleotide used as a probe or primer. Thus, polynucleotides with high homology (e.g., higher than about 70% or more) are encompassed by the prostacyclin synthase gene used in the present invention.

Herein, the phrase “high homology” refers to a identity of at least 50% or more, preferably 70% or more, more preferably 80% or more, and much more preferably 90% or more (e.g., 95% or more). The similarity of amino acid or polynucleotide sequences can be determined based on the BLAST search algorithm (Karlin and Altschul (1993) Proc. Natl. Acad. Sci. U.S.A. 90: 5873-7). Programs, such as BLASTN and BLASTX, have been developed based on this algorism (Altschul et al. (1990) J. Mol. Biol. 215: 403-10). Detailed methods for analyzing sequence similarity based on the algorism can be found at http://www.ncbi.nlm.nih.gov.

Furthermore, polynucleotides encoding proteins with modified amino acid sequence from the natural prostacyclin synthase are also included in the prostacyclin synthase gene. Such modification of amino acid sequence can be accomplished by, for example, site directed mutagenesis (Ausubel et al. edit. “Current Protocols in Molecular Biology”, 1987, John Wiley & Sons, Section 8.1-8.5). Proteins with artificially modified one or more amino acid residues from the original sequence may be obtained according to this method. Amino acids to be mutated in such mutants are generally within 10 amino acids, preferably 6 amino acids or less, and more preferably 3 amino acids or less.

To conserve the biological function of the prostacyclin synthase, it is preferable to mutate the amino acid residue into one that allows the properties of the amino acid side-chain to be conserved. The properties of amino acids are generally classified into: (1) hydrophobic amino acids (alanine, isoleucine, leucine, methionine, phenylalanine, proline, tryptophace, tyrosine and valine); (2) hydrophilic amino acids (arginine, aspargines, aspartic acid, cysteine, glutamic acid, glutamine, glycine, histidine, lysine, serine and threonine); (3) amino acids having aliphatic side-chain (alanine, glycine, isoleucine, leucine, phenylalanine and valine); (4) amino acids having hydroxyl group-containing side chain (serine, threonine and tyrosine); (5) amino acids having sulfur atom-containing side chain (cysteine and methionine); (6) amino acids having carboxylic acid- and amide-containing side chain (aspargine, aspartic acid, glutamic acid and glutamine); (7) amino acids having base-containing side chain (arginine, histidine and lysine); and (8) amino acids having aromatic-containing side chain (histidine, phenylalanine, tyrosine and tryptophane).

Examples of proteins having one or more amino acids added thereto include, but are not limited to, fusion proteins. For example, to prepare a polynucleotide encoding a fusion protein, a first DNA encoding a prostacyclin synthase and a second DNA encoding another protein or polypeptide are linked in frame. The protein or polypeptide that can be fused to the prostacyclin synthase is not limited to any specific protein or polypeptide. For example, a signal sequence directing secretion of the protein may be attached.

The prostacyclin synthase gene used in the present invention can be obtained based on known sequence information from, for example, mammalian cDNA or genomic libraries. For instance, a cDNA or genomic library is screened using a probe (e.g., antibody against the prostacyclin synthase or a oligonucleotide consisting of about 20 to 80 base pairs). Procedures for such screening can be performed according to general methods described, for example, in Sambrook et al. “Molecular Cloning: A Laboratory Manual” (New York, Cold Spring Harbor Laboratory Press, 1989) Section 10-12 and. Ausubel et al. “Current Protocols in Molecular Biology” (John Wiley & Sons, 1987) Section 6.3-6.4. Alternatively, PCR may be conducted to obtain genes encoding the prostacyclin synthase (see, e.g., Sambrook et al. (1989) supra, Section 14 and Ausubel et al. (1987) supra, Section 6.1-6.4). Further, according to needs the obtained polynucleotides may be modified according to conventional techniques as needed. Alternatively, the prostacyclin gene may be chemically synthesized.

Preferably, the prostacyclin synthase gene of the present invention is inserted into a vector which ensures expression of the gene in vivo, and which may be administered to the subject. The vector may be plasmid, phage, cosmid, virus and other conventionally used vectors. Those skilled in the art can prepare various vectors adapted for the present invention according to general methods (Ausubel et al. edit. “Current Protocols in Molecular Biology”, 1987, John Wiley & Sons; Sambrook et al. edit. “Molecular Cloning: A Laboratory Manual”, 1989, Cold Spring Harbor Laboratory).

The expression vector may further comprise regulatory genes, such as a promoter, enhancer and/or terminator, that are required for the expression of the prostacyclin synthase gene. The promoter, terminator and such used to regulate the expression of the present polynucleotide may be selected from known regulatory sequences. Namely, homogeneous as well as heterogeneous regulatory sequences can be used for the expression of the polynucleotide in the present invention. Furthermore, markers such as antibiotic resistance markers may be used for the expression vector as needed. Any commercially available expression vectors may be used in the present invention. However, it is preferred to delete unnecessary sequences from the vector.

Various methods for introducing nucleic acids into subjects are known in the art (Friedman (1989) Science 244: 1275-81). These methods can be categorized into methods using non-virus vectors and methods using virus vectors (Supplemental Experimental Medicine “Basic methods for gene therapy (Japanese)” 1996, Yodosha; Supplemental Experimental Medicine “Experimental Methods for Gene transfer & Expression Analysis (Japanese)” 1997, Yodosha; Japanese Society of Gene therapeutics edit. “Handbook for gene therapy development and research” 1999, NTS).

For example, in vitro transfection of the polynucleotide into a mammalian cell can be carried out with non-viral vectors by electroporation, liposome method (e.g., internal type liposome, electrostatic type liposome, Hemaggulutinating virus of Japan (HVJ)-liposome, HVJ-AVE liposome, etc.), microinjection, cell fusion, DEAE-dextran method, calcium phosphate transfection, naked plasmid method, methods using particle gun and so on.

Favorable in vivo gene transfection methods include methods using virus vectors. For example, detoxified DNA or RNA virus, such as retrovirus, adenovirus, herpesvirus, vaccinia virus, poxvirus, adeno-associated virus, poliovirus, HIV and HVJ (Adolph “Virus genome method”, 19.96, CRC Press) may be used as vectors for inserting the polynucleotide of the present invention.

Gene therapy according to the present invention can be conducted according to the in vivo or the ex vivo method (see, e.g., Japanese Society of Gene therapeutics edit. “Handbook for gene therapy development and research” 1999, NTS).

The effective dose and toxicity of a polynucleotide can be determined via cell culture assay or using an adequate animal model. Animal models can be also used to determine the administration route and the range of concentration. Based on the result using the animal model, one skilled in the art can determine a preferred dose, administration route and such for human subjects. The therapeutic coefficient of a polynucleotide is expressed as ED50/LD50 and polynucleotides with higher value are preferred for use. The dose of the prostacyclin synthase gene depends on the administration route and form, the susceptibility, symptom, weight, sex, age and such of the patient, however it is administered to an adult (calculated as a body weight of 60 kg) once every few days or few months at a range of about 1 μg to about 50 mg, preferably about 10 μg to about 10 mg, and more preferable about 50 μg to about 5 mg.

In some cases, it may be preferable to administer the prostacyclin synthase gene of the present invention with agents that target the objective cell to introduce the gene. For example, such agents are exemplified by antibodies specific to cell surface membrane proteins or target cells, or ligands against receptors on the target cell. When liposomes are used, it is preferred for ready targeting and/or intake to use proteins that bind to the cell surface membrane protein relating to endocytosis (e.g., capsid proteins tropic to a specific cell type or fragments thereof), proteins localized into the cell, proteins enhancing half-life in the cell, and such. Methods utilizing endocytosis is described, for example, by Wu et al. (J. Biol. Chem. 262: 4429-4432 (1987)) and Wagner et al. (Proc. Natl. Acad. Sci. U.S.A. 87: 3410-4 (1990)). Furthermore, method for marking genes and gene therapy techniques are described in detail, for example, by Anderson et al. (Science 256: 808-13 (1992)).

The present invention further provides a method for treating or preventing inflammatory disorders by administering a therapeutically effective amount of prostacyclin synthase into a subject.

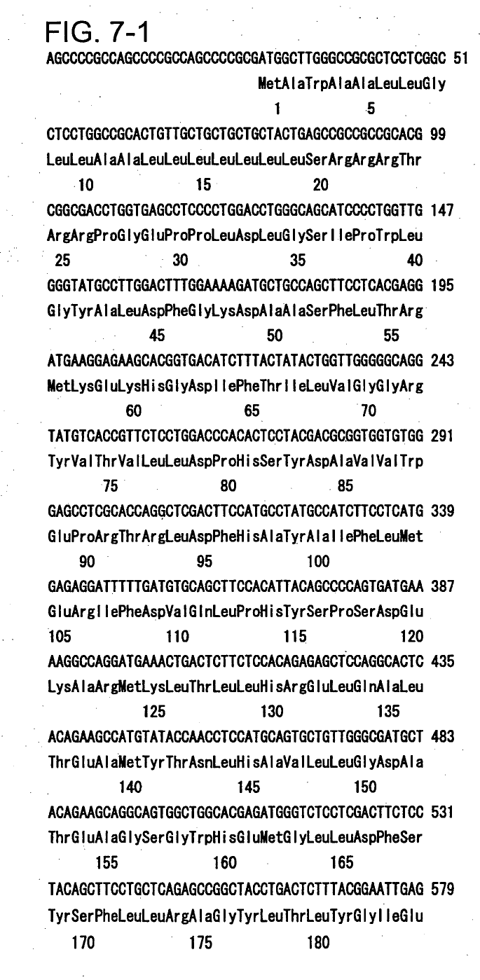

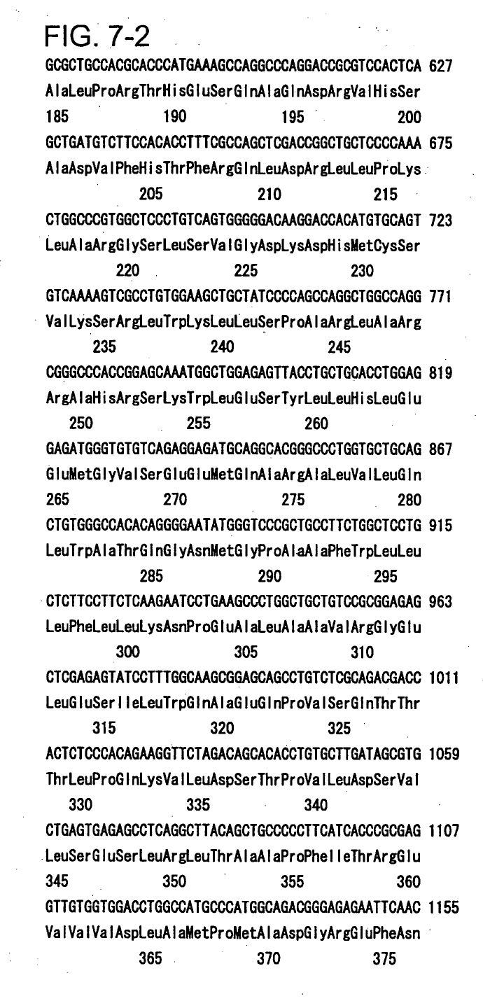

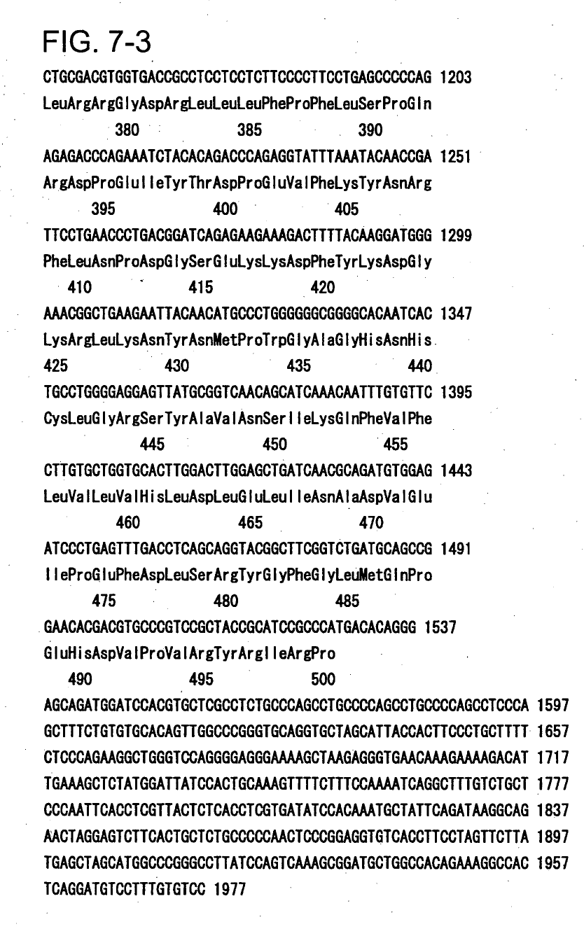

Herein, the phrase “prostacyclin synthase” refers to any kind of synthase that catalyzes the reaction of prostaglandin endoperoxide to PGI₂ homologues. A preferred example of the enzyme includes those derived from human having the amino acid sequence depicted in FIG. 7 (SEQ ID NO: 2). Enzymes having the amino acid sequence of SEQ ID NO: 2 can be obtained, for example, using antibodies against the prostacyclin synthase.

However, the present invention is not restricted to the human prostacyclin synthase. Enzymes that can be used in the present invention encompass those that are functionally equivalent to the protein having the amino acid sequence of SEQ ID NO: 2. Such functionally equivalent proteins include those encoded by any of the prostacyclin synthase genes described above.

To prepare the prostacyclin synthase via genetic recombination techniques, a DNA encoding the synthase is inserted into an expression vector so that the DNA is expressed under the control of the expression regulatory region (e.g., enhancer, promoter, etc.). Next, the expression vector is transformed into a host cell to express the synthase. For instance, when a mammalian cell is used as the host cell, the DNA encoding the synthase is linked to a regulatory sequence that functions in the mammalian cell. Such regulatory sequence includes human cytomegalovirus early promoter/enhancer, promoter/ enhancer of human elongation factor 1a (HEF1a)(Mizushima et al., Nucleic Acids Res. (1990) 18: 5322) and promoters/enhancers derived from retroviruses, polyoma viruses, adeno viruses and simian virus 40 (Mulligan et al., Nature (1979) 277: 108). Then, poly A signal is functionally linked downstream of the DNA encoding the synthase to construct an expression vector for the synthase.

Alternatively, E. coli and such bacterial cells may be used as the host cell. When E. coli is used, promoter, signal sequence for secretion and gene encoding a prostacyclin synthase is functionally linked together to express the synthase. For example, promoters such as lacZ promoter (Ward et al., Nature (1998) 341: 544-546;Ward et al., FASEB J. (1992) 6:2422-2427) and araB promoter (Better et al., Science (1988) 240: 1041-1043) are used for the expression.

Furthermore, to secrete the expressed synthase into the periplasm, for example, pelB signal sequence (Lei et al., J. Bacteriol. (1987) 16: 4379) may be used. Moreover, replication origins derived from SV40, polyoma virus, adeno virus, bovine papiloma virus and such may be used in the expression vector. To enhance the copy number in the host cell, it is preferred to combine a selection marker in the vector. Such selection markers are exemplified by genes such as aminoglycoside transferase gene, thymidine kinase gene, E. coli xanthine-guanine phosphoribosyl transferase gene and dihydrofolate dehydrogenase.

Any expression vector may be used for the expression of the prostacyclin synthase used in the present invention. For example, those derived from mammals, such as pEF and pCDM8; those derived from insect cells, such as pBacPAK8; those derived from plants, such as pMH1 and pMH2; those derived from animal virus, such as pHSV, pMV, pAdexLcw and such; yeast derive expression vectors, such as pZIpneo, pNV11 and SP-Q01; Bacillus subtilis derived expression vectors, such as pPL608 and pKTH50; and E. coli derived expression vectors, such as pQE, pGEAPP, pGEMEAPP and pMALp2.

The expression vector constructed as above may be introduced into a host cell by calcium phosphate transfection (Virology (1973) 52: 456-67), electroporation (EMBO J. (1982) 1: 841-5), etc.

Any arbitrary in vitro or in vivo production system may be employed for the production of the prostacyclin synthase used in the present invention. The in vitro production system includes those using eucaryotic cells or prokaryotic cells.

Production systems using eucaryotic cells include those using animal cells, plant cells and fungal cells. Systems using animal cells like mammalian cell, such as CHO (J. Exp. Med. (1995) 108: 945), COS, myeloma, baby hamster kidney, HeLa and Vero; amphibian cells, such as Xenopus oocyte (Valle et al., Nature (1981) 291: 358-40); and insect cells, such as sf9, sf21 and Tn5 are known in the art. Particularly preferred CHO cells include DHFR gene-deficient dhfr-CHO (Proc. Natl. Acad. Sci. U.S.A. (1980) 77: 4216-20) and CHO K-1 (Proc. Natl. Acad. Sci. U.S.A. (1968) 60: 1275). Yeasts of the genus Saccharomyces and filamentous fungi of the genus Aspergillus are known as fungal cells that can be used for the production of proteins. Bacterial cells of E. coli and Bacillus subtilis may be mentioned as prokaryotic cells that can be used for the production of proteins.

These cells are transformed by the aforementioned expression vectors and cultured in vitro to obtained the objective prostacyclin synthase. Cultivation of cells can be performed according to conventional methods. For example, DMEM, MEM, RPMI1640, IMDM and such may be used as the culture medium, and these may also be supplemented with serum (e.g., FCS). One of ordinary skill in the art can determine the condition for the culture that differs depending on the used host cell. In the culture, the culture may be replaced, aerated, stirred and such as needed.

Alternatively, as in vivo production systems, those using animals and plants can be mentioned. For example, systems enabling recovery of the enzyme from milk (e.g., goat (Ebert et al., Bio/Technology (1994) 12: 699-702)) or systems using insects like silkworms (Susumu et al., Nature (1985) 315: 592-4) are preferred.

The produced enzyme used for the present invention may be isolated as a substantially pure homogeneous protein. Isolation and/or purification of the prostacyclin synthase can be performed following conventional protein purification methods and is not restricted in any way. For example, techniques such as chromatography, filtration, ultrafiltration, salting out, solvent precipitation, solvent extraction, distillation, immuno precipitation, electrophoresis, dialysis, recrystallization, etc. may be used alone or in combination. Chromatography such as affinity chromatography, ion exchnage chromatography, hydrophobic chromatography, gel filtration, absorption chromatography, reverse phase chromatography and such are known in the art, and can be used (Daniel R. Marshak et al. ed. “Strategies for Protein Purification and Characterization: A Laboratory Course Manual”, Cold Spring Harbor Laboratory Press, 1996). These chromatographies can be performed using HPLC or FPLC. To attain ready purification of the enzyme, the enzyme may be modified with arbitrary peptides (e.g., tags) that may be deleted with peptidase after the purification.

In the present invention, the prostacyclin synthase is administered alone or formulated as a preparation to a subject. The enzyme may be administered orally, parenterally, subcutaneously, interdermally, intravascularly, etc. Moreover, for example, the agonist may be solubilized in a sterile pharmaceutically acceptable solution, such as distilled water, phosphate buffered saline (PBS), etc., as needed. In addition, the solution may comprise buffers, colorants, corrigents, diluents, disintegrators, emulsifiers, excipients, extenders, fragrances, preservatives, solubilizers, stabilizers, sweetners, etc. Using such additives, the enzyme may be formulated into capsules, elixirs, emulsions, granules, injections, pills, powders, solutions, suspensions, syrups, tablets, troches, etc. as needed.

The dose of the prostacyclin synthase depends on the administration route, and the weight, age, symptom, sex and such of the subject to be treated; but those skilled in the art can be determine a preferable dose. However, generally, a dose of about 1 μg to 100 mg or less per day may be administered to an adult (body weight 60 kg).

Inflammatory disorders that can be treated according to the present invention are interleukin-13 (IL-13) mediated ameliorable inflammatory disorders, including rheumatoid arthritis, rheumatoid spondylitis, osteoarthritis, gouty arthritis, inflammatory bowel disease, Crohn's disease, Guillain-Barre syndrome, schleroderma, fibrosis, dermatitis, psoriasis, angioedema, eczematous dermatitis, hyperproliferative skin disease, inflammatory skin conditions, glomerulonephritis, nephritis, vascular inflammation, atherosclerosis, angitis, phlebitis, arteritis, aorititis, post PTCA restenosis, post by-pass surgery restenosis, transplantation rejection, anaphylaxis, sepsis, thrombosis, ischemia/reperfusion injury and autoimmune diseases are treated. Exemplary transplantation rejections that can be treated according to the present method include renal allograft rejection, cardiac allograft rejection and transplantation-associated vasculopathy.

Subjects to be treated according to the present invention include mammals, such as dogs, cats, mice, rats, sheep, cows, pigs, monkeys and so on. However, human subjects are particularly preferred.

The following examples are presented to illustrate the present invention and to assist one of ordinary skill in making and using the same. The examples are not intended in any way to otherwise limit the scope of the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. Any patents, patent applications and publications sited herein are incorporated by reference.

### BEST MODE FOR CARRING OUT THE INVENTION

1. Methods

(1) Reagents

cPGI was purchased from Cayman Chemica Co. (Ann Arbor, Mich.). Recombinant human TNF-α and IL-1β were obtained from Peprotech Inc. (Rocky Hill, N.J.). Anti-IL-13 monoclonal antibody and control IgG were products of Santa Cruz Biotechnology (Santa Cruz, Calif.). Dulbecco's modified Eagle's medium (DMEM) was purchased from Gibco-Invitrogen (Gaithersburg, Md.), and EGM-2 growth medium and a mixture of growth factors were from Sanko Junyaku Co., Ltd. (Tokyo, Japan).

(2) Cell Culture

Primary cultured human umbilical vein endothelial cells (HUVEC) and human aortic endothelial cells (HAEC) were obtained from Kurabo Co. Ltd. (Tokyo, Japan) and were cultured in EGM-2 growth medium supplemented with 2% fetal bovine serum (FBS) and a mixture of growth factors according to the supplier's instructions. Total RNA from the cells in culture was prepared with Isogen (Nippon Gene, Tokyo, Japan). Human embryonic kidney HEK-293 cells were cultured in DMEM containing 10% FBS, 2 mM L-glutamine, 100 U/ml penicillin and 100 mg/ml streptomycin as described previously (Hatae et al., J. Biol. Chem. (2001) 276: 46260-7). For expression of wild-type PGIS (PGISwt), inactive mutant PGIS (PGISC441A), wild-type PPARδ (PPARδwt), dominant negative mutant PPARδ (PPARδdn) (Hatae et al., J. Biol. Chem. (2001) 276: 46260-7), wild-type CREB (CREBwt) and dominant negative mutant CREB (CREBdn), plasmid vectors pCMV/PGISWT (Hatae et al., FEBS Lett. (1996) 389:268-72), pCMV/PGISC441A (Hatae et al., FEBS Lett. (1996) 389: 268-72), pcDNAPPARδwt (Hatae et al., J. Biol. Chem. (2001) 276: 46260-7), pcDNAPPARδ L431A/G434A (Hatae et al., J. Biol. Chem. (2001) 276: 46260-7), pCMVCREB (Clontech, Palo Alto, Calif.) and pCMVCREB133 (Clontech) were used, respectively. HEK-293 cells were transfected with DNA using lipofectAMINE (Invitrogen, Carlsbad, Calif.) as described previously (Hatae et al., J. Biol. Chem. (2001) 276: 46260-7). Human vascular endothelial cells were transfected with TransIT LT-1 (PanVera Co., Madison, Wis.). Transfections were performed at a ratio of 1 μg of DNA to 3.0 μl of the reagents, and cells were incubated in serum-free EGM-2 at 37° C. for 5 h. Subsequently, cells were grown in fresh EGM-2 containing 2% FBS and a mixture of growth factors for 12 h before analysis. For reporter assay, human vascular endothelial cells (1.2×10⁴ cells/cm²) were cotransfected with 0.2 μg of the PPREx3-luciferase reporter vector and 0.1 μg of β-galactosidase expression vector by use of TransIT LT-1. After 17 h of transfection, cells were washed with PBS, incubated in EGM-2 containing 2% FBS and cPGI (0-100 μM) for 24 h at 37° C., and subjected to luciferase assay as described previously (Hatae et al., J. Biol. Chem. (2001) 276: 46260-7). The luciferase activity of the extract was normalized to β-galactosidase activity.

(3) cDNA Microarray Analysis

HEK-293 cells (1.2×10⁴ cells/cm²) were transfected with expression vector for PGISwt or PGISC441A using LipofectAMINE (Invitrogen) and maintained in DMEM containing 10% FBS, 2 mM L-glutamine, 100 U/ml penicillin and 100 mg/ml streptomycin for 36 h. Poly(A+) mRNA (1.5 μg) was prepared from3×10⁶ cells using Isogen (Nippon Gene) and Oligotex-dT30 super mRNA purification kit (Takara, Tokyo, Japan). Samples were analyzed by gene expression microarray (GEM) DNA tip containing 9,182 human cDNA clones (HUMAN UniGEM V Ver2.0, IncyteGenomics, Palo Alto, Calif.). Conversion of poly(A+) mRNA to cDNA with incorporation of Cy3- or Cy5-labeled deoxynucleotide-triphosphates (dNTPs), hybridization to arrays coated on glass, quality control and normalization were performed by IncyteGenomics. All data analyses were performed using an average fold change of two assays, and data were expressed as the ratio of PGISwt to PGISC441A.

(4) Cloning of IL-13 Gene and Construction of Reporter Vectors

Promoter sequences of human IL-13 gene containing PPRE and GATA elements were isolated from human leukocyte genomic DNA by polymerase chain reaction (PCR) with primers A (5′-CAGAGAGGGTGGGAATGACG-3′ (SEQ ID NO: 3); sense) and D (5′-GTGCCAACAGGATTGAGGAGCGGA-3′ (SEQ ID No: 4); antisense) for the 1.3 kbp fragment, and B (5′-GTCGGGATTTTATGAATGAA-3′ (SEQ ID NO: 5); sense) and D for the 0.4 kbp fragment. Each fragment was inserted into the MluI-XbaI site of pGL3-promoter vector (Promega, Madison, Wis.). Human vascular endothelial cells (1.2×10⁴ cells/cm²) were cotransfected with 0.2 μg of IL-13-luciferase reporter vector, 0.1 μg of β-galactosidase expression vector and 0.7 μg of expression vector for PPARδwt, PPARδdn, CREBwt, CREBdn or mock vector by use of TransIT LT-1. After 17 h of transfection, cells were incubated in fresh EGM-2 containing 2% FBS. After another 24 h, the cells were washed with PBS, and cultured in EGF-2 containing 2% FBS with 10 μM cPGI for 24 h to quantify luciferase and β-galactosidase activities as described previously (Hatae et al., J. Biol. Chem. (2001) 276: 46260-7). The luciferase activity of the extract was normalized to β-galactosidase activity.

(5) Analysis of IL-13 and COX-2 mRNA

For analysis of IL-13, human vascular endothelial cells (1.2×10⁴ cells/cm²) were stimulated with cPGI (10 μM) for 12 h, and total RNA was prepared from stimulated cells with Isogen (Nippon Gene). For analysis of COX-2, human vascular endothelial cells (1.2×10⁴ cells/cm²) were pretreated with TNF-α (1 ng/ml) and IL-1b (20 ng/ml) for 1 h, and then treated with cPGI (0, 10 μM), anti-human IL-13 monoclonal antibody (0, 0.5 μg/ml) and control IgG (0, 0.5 μg/ml) for 12 h to prepare total RNA. The total RNA (0.5 μg) was used for analysis by SuperScript RT-PCR system (Invitrogen) according to the manufacturer's instructions. The oligonucleotide primers used were IL-13-s (5′-GACTGCAGTCCTGGCTCTTGC-3′ (SEQ ID NO: 6); sense) and IL-13-as (5′-TGAGTCCACAGCTGAGATGCC-3′ (SEQ ID NO: 7); antisense) for human IL-13, β-actin-s (5′-GTGGGCCGCTCTAGGCACCA-3′ (SEQ ID NO: 8); sense) and β-actin-as (5′-CGGTTGGCCTTAGGGTTCAGGGGGG-3′ (SEQ ID NO: 9); antisense) for human β-actin, and COX-2-s (5′-GGGTTGCTGGGGGAAGAAATGTG-3′ (SEQ ID NO: 10); sense) and COX-2-as (5′-GGTGGCTGTTTTGGTAGGCTGTG-3′ (SEQ ID NO: 11); antisense) for human COX-2. For each PCR amplification, 1 μl of cDNA was used in a 50 μl reaction. Reverse transcription-PCR (RT-PCR) for β-actin (25 cycles) was used as an internal control for mRNA abundance. For IL-13 and COX-2 genes, the number of cycles ranged from 25 to 45. The efficiency of the RT-PCR reaction for each gene did not plateau, and the numbers of cycles used in these experiments were kept to a minimum. Samples were analyzed by gel electrophoresis, and bands were revealed by staining gels with ethidium bromide. Samples were normalized based on equivalent expression of β-actin.

(6) IL-13 and PGI₂ Assays

Human vascular endothelial cells (1.2×10⁴ cells/cm²) were cultured in EGM-2 containing 2% FBS and several concentrations of cPGI with or without TNF-α for 24 h. Incubation media were collected and stored at −80° C. before assay. Amounts of IL-13 were measured with human IL-13 ELISA system (Amersham Pharmacia Biotech, Buckinghamshire, UK) and amounts of PGI₂ were measured as the amounts of 6-keto-PGI_{1α}, a stable metabolite of PGI₂, with 6-keto-PGI_{1α} ELISA kit (Cayman Chemical Co., Ann Arbor, Mich.) according to the manufacturers' instructions.

(7) Adherence Assays

HUVEC (2.5×10⁴ cells/cm²) were cultured in 12-well plates. After 24 h, confluent monolayers were used for adherence assays. Monolayers were preincubated in EGM-2 containing 2% FBS, 0 or 10 ng/ml TNF-α, 0 or 10 ng/ml IL-13 and 0 or 10 μM cPGI with or without anti-IL-13 monoclonal antibody (0.5 μg/ml) at 37° C. for 0 to 24 h. Human monocyte leukocyte U937 cells were cultured in RPMI 1640 (Gibco-Invitrogen) containing 10% FBS, 100 U/ml penicillin and 100 mg/ml streptomycin , labeled with 2.5 μM calcein-AM (Molecular Probes, Eugene, Oreg.) at room temperature for 45 min, washed and resuspended in serum-free EGM-2. The calcein-labeled U937 cells (8×10⁴ cells/well) were added to pretreated endothelial cells and incubated with cPGI (0, 10 μM), recombinant IL-13 (0, 10 ng/ml) and anti-IL-13 monoclonal antibody (0, 0.5 μg/ml). U937 cells were allowed to adhere to pretreated endothelial cells for 30 min at 37° C. Adherence was assessed with a fluorescence image analyzer (FLA-2000; Fujifilm, Tokyo, Japan). Plates were scanned before and after washing for total and adherent cells, respectively, and relative adherence was calculated (fold of control).

(8) RNA Interference (RNAi) with Small Interfering RNAs (siRNAs)

siRNA duplexes with the following sense and antisense sequences were made: PGIS1, 5′-GGAAACGGCUGAAGAAUUAUU-3′ (SEQ ID NO: 12; sense) and 5′-UAAUUCUUCAGCCGUUUCCUU-3′ (SEQ ID NO: 13; antisense); PGIS2, 5′-GCGGAGAGCUCGAGAGUAUUU-3′ (SEQ ID NO: 14; sense) and 5′-AUACUCUCGAGCUCUCCGCUU-3′ (SEQ ID NO: 15; antisense); PGIS3, 5′-CCGGCUACCUGACUCUUUAUU-3′ (SEQ ID NO: 16; sense) and 5′-UAAAGAGUCAGGUAGCCGGUU (SEQ ID NO: 17; antisense); and PGIS4, 5′-UCAACAGCAUCAAACAAUUUU-3′ (SEQ ID NO:, 18; sense) and 5′-AAUUGUUUGAUGCUGUUGAUU-3′ (SEQ ID NO: 19; antisense). siRNAs were synthesized and annealed by Dharmacon Research Inc. (Lafayette, CO). Control non-silencing siRNA, a duplex of sense (5′-UUCUCCGAACGUGUCACGUdTdT-3′; SEQ ID NO: 20) and antisense (5′-ACGUGACACGUUCGGAGAAdTdT-3′; SEQ ID NO: 21) sequences, was purchase from Qiagen (Valencia, Calif.). The cells were initially transfected with 200 pmol per well of the annealed RNA mixture or the same amount of control siRNA using LipofectAMINE2000 (Invitrogen), and 12 h later secondly transfected with the same amount of siRNAs using HVJ-liposome transfection kit (GenomeONE; Ishihara Sangyo, Osaka, Japan) according to the manufacturers' instructions. The cells were retransfected with the siRNAs every 3 days using LipofectAMINE2000 and HVJ-liposome, and used for analyses 10 days after transfection. Microsomal fractions of the cells were prepared and analyzed by immunoblotting with anti-PGIS P4 polyclonal antibody as described previously (Hatae et al., J. Biol. Chem. (2001) 276: 46260-7). The cells treated with siRNAs for 10 days were cultured in fresh EGM-2 containing 2% FBS and TNF-α (0, 10 ng/ml). with or without cPGI (10 μM) for 24 h. The incubation media were collected, and amounts of 6-keto-PGI_{1α} or IL-13 were measured with ELISA kit.

2. Results

(1) PPARδ Activation with Prostacyclin Up-regulates Expression of IL-13

In order to identify the downstream mediators of intracellular PGI₂-PPARδ signaling pathway, a cDNA microarray analysis of human cells expressing intracellular PGI₂ was performed. Based on precious studies of the inventors (Hatae et al., J. Biol. Chem. (2001) 276: 46260-7), a human embryonic kidney cell line (HEK-293) transfected with the expression vector for wild-type PGIS (PGISwt) was used to achieve endogenous expression of intracellular PGI₂. An expression vector for enzymatically inactive PGIS (PGISC441A) was used as control.

TABLE 1
Typical genes significantly up-regulated by intracellular
PGI₂ formed by PGIS in HEK-293 cells

| Differential | | Accession |
|---|---|---|
| expression | Gene Name | No. |
| +11.0 | IL-13 | NM_002188 |
| +7.0 mal | T-cell differentiation protein | NM_022439 |
| +3.7 | 2′,5′-oligoadenylate synthetase 1 (40-46 kD) | AL582281 |
| +3.5 | heat shock 70 kD protein 1A | G674024 |
| +3.2 | 3-hydroxy-3-methylglutaryl-coenzyme A synthase 1 | AW117731 |
| | (soluble) | |
| +2.9 | isopentenyl-diphosphate delta isomerase | BF698441 |
| +2.8 | Sjogren syndrome antigen | BF897247 |
| +2.8 | retinoic acid- and interferon-inducible protein | NM_012420 |
| | (58 kD) | |
| +2.7 | guanylate binding protein 1, interferon-inducible, | BF913224 |
| | 67 kD | |
| +2.7 | interferon-stimulated transcription factor 3, gamma | NM_006084 |
| | (48 kD) | |
| +2.7 | butyrophilin, subfamily 3, member A2 | AA482308 |
| +2.5 | aldehyde dehydrogenase 1 family, member A1 | AI050734 |
| +2.5 | selectin P (granule membrane protein 140 kD, antigen | NM_003005 |
| | CD62) | |

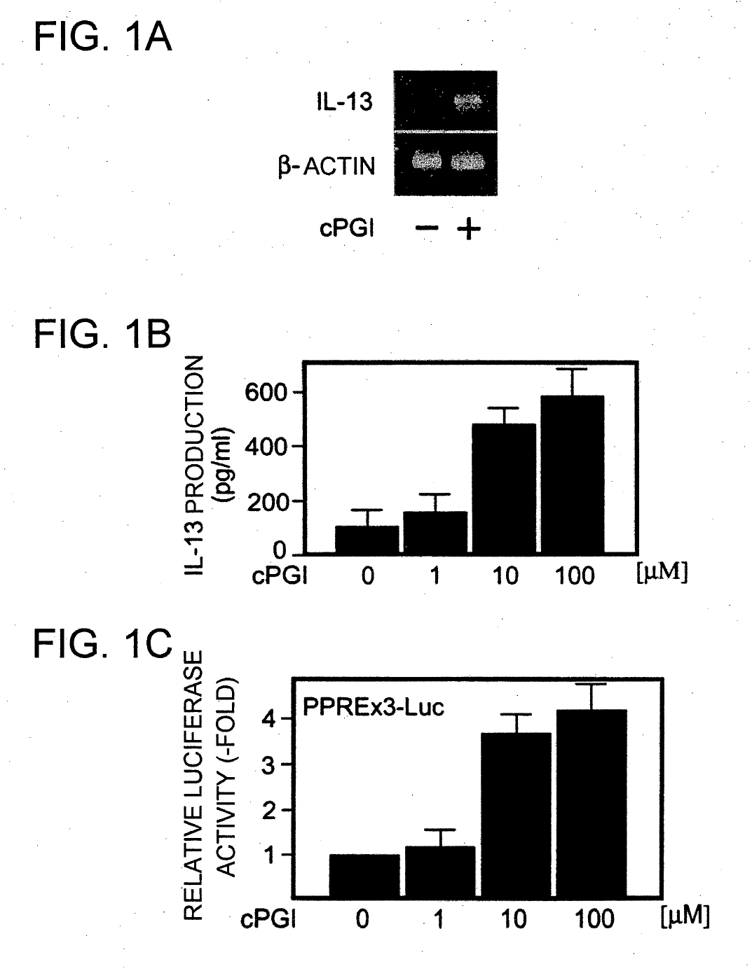

As show in Table 1, the IL-13 gene was up-regulated in the cells producing intracellular PGI₂ with the greatest fold change. In order to confirm that PGI₂ had a similar effect on the expression of IL-13 in cells from the cardiovascular system, HUVEC were treated with 10 μM cPGI, a membrane-permeable stable analog of PGI₂, and RT-PCR with specific oligonucleotide primers was performed on mRNA extracted from the cells. As shown in FIG. 1A, apparent induction of IL-13 mRNA expression was observed. These results demonstrate that the expression of IL-13 gene is induced in the treated cells. Moreover, IL-13 protein in the culture medium was detectable by ELISA (FIG. 1B). A dose-dependent correlation between cPGI-dependent activation of luciferase activities of reporter vector containing PPRE and production of IL-13 protein was observed (FIGS. 1B and 1C). Similar results were obtained when primary cultured human aortic endothelial cells (HAEC) were treated. with CPGI under the same conditions (data not shown).

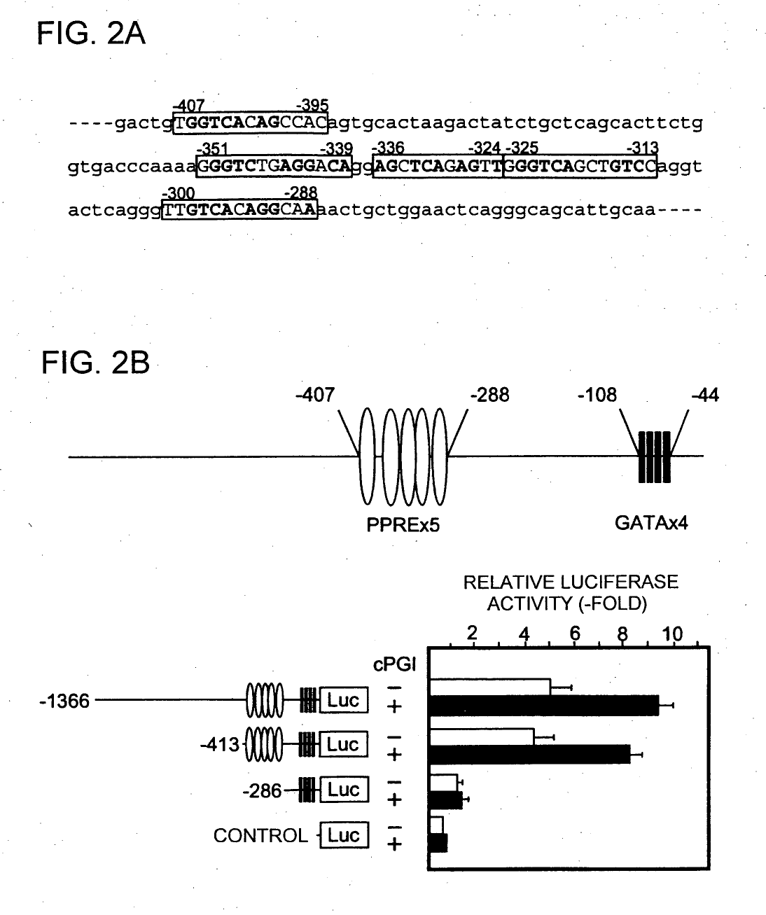

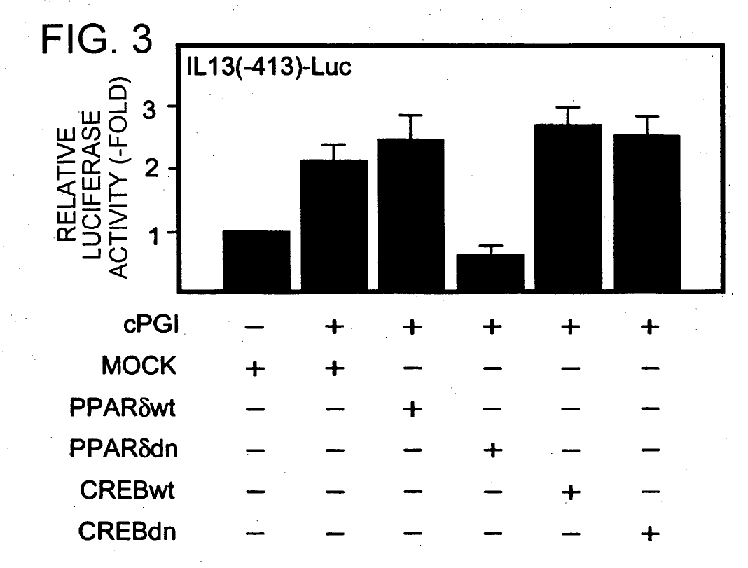

To clarify the molecular bases of the mechanism of PGI₂-dependent induction of IL-13, the promoter region of the human IL-13 gene was searched for PPRE consensus sequences, revealing five PPRE consensus sequences at positions of −407, −351, −336, −325 and −300 (FIG. 2A). When reporter vectors containing the PPRE consensus region of the IL-13 promoter were transfected into HUVEC, their luciferase activities were significantly increase by cPGI treatment (FIG. 2B). On the contrary, the luciferase activities of vectors from which the PPRE consensus sequence region had been deleted were not increased by cPGI. It has been reported that GATA sequence from −108 to −44 are important for the regulation of human IL-13 gene expression, and that cAMP increases IL-13 gene expression through p38 activation and phosphorylation of GATA protein (Hatae et al., FEBS Lett. (1996) 389: 268-72). However, the luciferase activity of a reporter vector containing GATA sequences without PPREs was increased only weakly by cPGI in the present study (FIG. 2B). Furthermore, cotransfection of dominant negative PPARδ (PPARδdn) suppressed cPGI-dependent increase of luciferase activity derived from the reporter vector that contained the PPRE consensus region of the IL-13 gene (FIG. 3). On the other hand, coexpression of dominant negative CREB (CREBdn) had no effect on the luciferase activity of reporter vector by cPGI under the same conditions. These data indicate that PGI₂-mediated induction of IL-13 protein and expression of the human IL-13 gene depend on the activation of PPARδ, and that the human IL-13 gene is a target of the PGI₂-PPARδ signaling pathway in human vascular endothelial cells.

(2) PPARδ-dependent Secretion of IL-13 Contributes to Inhibition of Proinflammatory Cytokine-mediated Adhesion Between Human Vascular Endothelial Cells and Monocytic Leukocyte U937 Cells

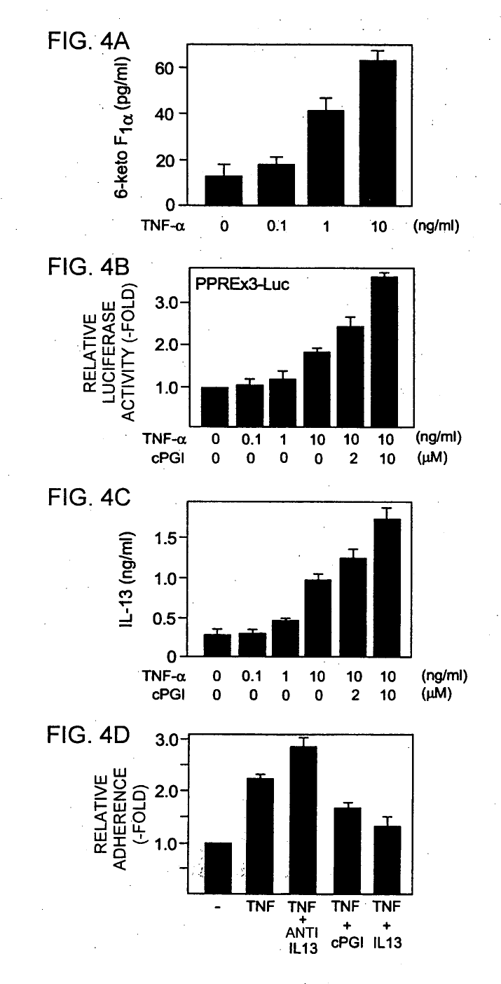

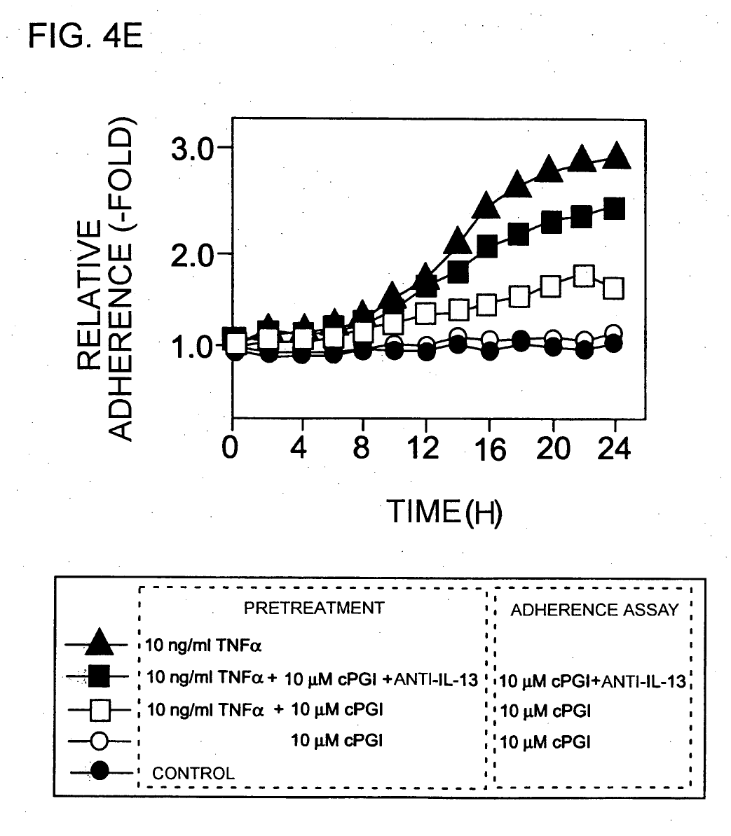

It is known that the inflammatory cytokine TNF-α induces production of endogenous PGI₂ in vascular endo-thelial cells via activation of the cyclooxygenase pathway (Langeler et al., Arterioscler Thromb. (1991) 11: 872-81). Therefore, in order to examine the physiological functions of the PGI₂-PPARδ-IL-13 pathway after activation with PGI₂, HUVEC were treated with human TNF-α. As shown in FIG. 4A, treatment of HUVEC with TNF-α significantly induced endogenous PGI₂ production. Moreover, the relative luciferase activity of a reporter vector containing PPRE (FIG. 4B) and production of IL-13 (FIG. 4C) increased. Exogenous addition of cPGI enhanced luciferase activity and production of IL-13 in a dose-dependent manner (FIGS. 4B and 4C).

It is also known that TNF-α increases adhesion between vascular endothelial cells and monocytes (Maier et al., Exp. Cell Res. (1993) 208: 270-4). Indeed, the treatment of HUVEC with TNF-α was also observed to increase adhesion between human monocytic leukocyte U937 cells and the endothelial cells (FIG. 4D, lanes 1 and 2). Interestingly, treatment of HUVEC with TNF-α and anti-IL-13 antibody significantly enhanced monocytic leukocyte-endothelial cell adhesion (FIG. 4D, lane 3). On the contrary, exogenous addition of cPGI or IL-13 inhibited the adhesion (FIG. 4D, lanes 4 and 5). For further characterization, HUVEC were incubated with TNF-α and cPGI with or without anti-IL-13 monoclonal antibody for 0 h to 24 h. As shown in FIG. 4E, monocytic leukocyte U937 cells adhered to endothelial monolayers in a time-dependent manner. On the contrary, control endothelial cells preincubated without TNF-α did not demonstrate increased adhesion between the monocytic cells and the endothelium. Pretreatment of HUVEC with TNF-α in the presence of cPGI dramatically inhibited adhesion. Furthermore, addition of anti-IL-13 monoclonal antibody significantly suppressed this cPGI-dependent inhibition of adhesion (FIG. 4E). These data suggest that IL-13 produced via activation of the PGI₂-PPARδ pathway has important roles in the regulation of the interaction between monocytic leukocyte U937 cells and endothelial cells induced by the proinflammatory cytokine TNF-α.

(3) PPAR8-dependent Secretion of IL-13 Elicited by cPGI Contributes to Inhibition of COX-2 Expression Induced by Proinflammatory Cytokines in Human Vascular Endothelial Cells

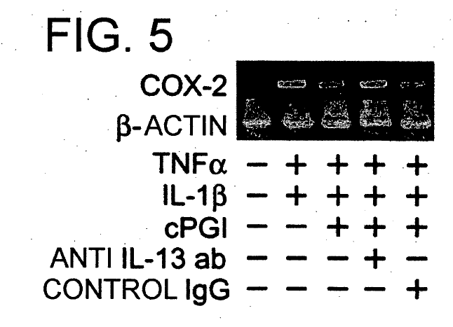

PGI₂ and other PGs are involved in a variety of physiological and pathological processes including inflammation. Moreover, COX-2 has important roles in the regulation of inflammation, and the gene of COX-2 is up-regulated by proinflammatory factors. To examine the effect of PGI₂/PPARδ-dependent production of IL-13 on the expression of COX-2 gene in vascular endothelial cells, COX-2 mRNA was measured by RT-PCR in HUVEC. As shown in FIG. 5, treatment of HUVEC with the proinflammatory cytokines TNF-α and IL-1 induced the expression of COX-2 mRNA. This inducible expression of COX-2 in endothelial cells was significantly inhibited by exogenous addition of cPGI (FIG. 5, lane 3). Furthermore, this cPGI-dependent suppression of COX-2 mRNA was significantly inhibited by the addition of anti-IL13 monoclonal antibody (FIG. 5, lane 4). On the contrary, the addition of control IgG had no effect on cPGI-dependent inhibition of COX-2 mRNA expression. These data show that exogenous addition of cPGI can regulate COX-2 gene expression via IL-13 production in the endothelium.

(4) Effects of Endogenous Production of PGI₂ and Exogenous cPGI on Control of Endogenous IL-13 Expression in Human Vascular Endothelial Cells

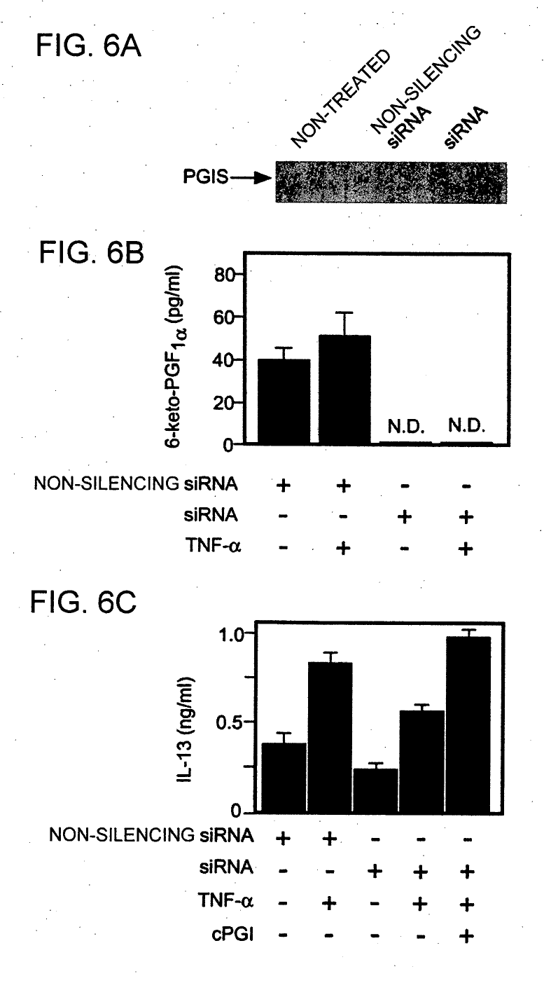

Reduced synthesis of endogenous PGI₂ is believed to play an important role in the development and progression of thrombosis, vascular inflammation and atherosclerosis. However, the relationship between endogenous production of PGI₂ by human vascular endothelial cells and IL-13 expression in the cells remains to be elucidated. RNAi-based silencing of the gene for PGIS was performed in HUVEC to determine the effects of endogenous PGI₂ production on the expression of IL-13 in human vascular endothelial cells. As shown in FIG. 6A, siRNA treatment of the cells suppressed the endogenous expression of PGIS, whereas control siRNA had no effect on PGIS expression. Moreover, the production of PGI₂, measured as 6-keto-PGF_{1α} was not detected in cells treated with siRNA (FIG. 6B). As shown in FIG. 6C, TNF-α related production of IL-13 decreased in cells lacking PGI₂ production. Furthermore, exogenous addition of cPGI significantly increased endogenous IL-13 expression in PGI₂-deprived cells. These results show that endogenous production of PGI₂ by human vascular endothelial cells contributes to IL-13 expression responding to stimulation of TNF-α in the cells, and that cPGI has the ability to control endogenous expression of IL-13 in endothelial cells with reduced capability for PGI₂ production.

While the invention has been described in detail and with reference to specific embodiments thereof, it will be apparent to one skilled in the art that various changes and modifications can be made therein without departing from the spirit and scope of the invention.

1. A method for treating or preventing inflammatory disorders, which comprises administering a therapeutically effective amount of peroxisome proliferator-activated receptor (PPAR) δ agonist into a subject.

2. The method of claim 1, wherein the PPARδ agonist is a prostacyclin analog or a carbaprostacyclin analog.

3. The method of claim 1, wherein the PPARδ agonist is penetrable into mammalian cells.

4. The method of claim 1, wherein the PPARδ agonist is a prostacyclin analog selected from the group consisting of prostacyclin (epoprostenol), beraprost, taprostene, nileprost and OP-2507, or pharmaceutically acceptable salts thereof.

5. The method of claim 1, wherein the PPARδ agonist is a carbaprostacyclin analog selected from the group consisting of carbaprostacyclin, iloprost, cicaprost, ciprostene, treprostinil and bonsentan, or pharmaceutically acceptable salts thereof.

6. The method of claim 1, wherein the inflammatory disorder is interleukin-13 (IL-13) mediated ameliorable inflammatory disorder.

7. The method of claim 1, wherein the inflammatory disorder is selected from the group consisting of rheumatoid arthritis, rheumatoid spondylitis, osteoarthritis, gouty arthritis, inflammatory bowel disease, Crohn's disease, Guillain-Barre syndrome, schleroderma, fibrosis, dermatitis, psoriasis, angioedema, eczematous dermatitis, hyperproliferative skin disease, inflammatory skin conditions, glomerulonephritis, nephritis, vascular inflammation, atherosclerosis, angitis, phlebitis, arteritis, aorititis, post PTCA restenosis, post by-pass surgery restenosis, transplantation rejection, anaphylaxis, sepsis, thrombosis, ischemia/reperfusion injury and autoimmune diseases.

8. The method of claim 1, wherein the inflammatory disorder is selected from the group consisting of vascular inflammation, atherosclerosis, inflammatory bowel disease and transplantation rejection.

9. The method of claim 1, wherein the inflammatory disorder is vascular inflammation.

10. The method of claim 1, wherein the inflammatory disorder is atherosclerosis.

11. The method of claim 1, wherein the inflammatory disorder is inflammatory bowel disease.

12. The method of claim 1, wherein the inflammatory disorder is transplantation rejection selected from the group consisting of renal allograft rejection, cardiac allograft rejection and transplantation-associated vasculopathy.

13. A method for treating or preventing inflammatory disorders, which comprises administering a prostacyclin synthase gene or a protein encoded by the gene into a subject.

14. The method of claim 13, wherein the prostacyclin synthase gene comprises the nucleotide sequence from the 28th base to the 1527th base of SEQ ID NO: 1.

15. The method of claim 13, wherein the inflammatory disorder is interleukin-13 (IL-13) mediated ameliorable inflammatory disorder.

16. The method of claim 13, wherein the inflammatory disorder is selected from the group consisting of rheumatoid arthritis, rheumatoid spondylitis, osteoarthritis, gouty arthritis, inflammatory bowel disease, Crohn's disease, Guillain-Barre syndrome, schleroderma, fibrosis, dermatitis, psoriasis, angioedema, eczematous dermatitis, hyperproliferative skin disease, inflammatory skin conditions, glomerulonephritis, nephritis, vascular inflammation, atherosclerosis, angitis, phlebitis, arteritis, aorititis, post PTCA restenosis, post by-pass surgery restenosis, transplantation rejection, anaphylaxis, sepsis, thrombosis, ischemia/reperfusion injury and autoimmune diseases.

17. The method of claim 13, wherein the inflammatory disorder is selected from the group consisting of vascular inflammation, atherosclerosis, inflammatory bowel disease and transplantation rejection.

18. The method of claim 13, wherein the inflammatory disorder is vascular inflammation.

19. The method of claim 13, wherein the inflammatory disorder is atherosclerosis.

20. The method of claim of claim 13, wherein the inflammatory disorder is inflammatory bowel disease.

21. The method of claim 13, wherein the inflammatory disorder is transplantation rejection selected from the group consisting of renal allograft rejection, cardiac allograft rejection and transplantation-associated vasculopathy.


SEQUENCE LISTING (22 sequences)

SEQ ID NO: 1 - DNA, 1977 bp, Homo sapiens
  CDS (28)..(1527)
agccccgccagccccgccagccccgcgatggcttgggccgcgctcctcggcctcctggccgcactgttgctgctgctgctactgagccgccgccgcacgcggcgacctggtgagcctcccctggacctgggcagcatcccctggttggggtatgccttggactttggaaaagatgctgccagcttcctcacgaggatgaaggagaagcacggtgacatctttactatactggttgggggcaggtatgtcaccgttctcctggacccacactcctacgacgcggtggtgtgggagcctcgcaccaggctcgacttccatgcctatgccatcttcctcatggagaggatttttgatgtgcagcttccacattacagccccagtgatgaaaaggccaggatgaaactgactcttctccacagagagctccaggcactcacagaagccatgtataccaacctccatgcagtgctgttgggcgatgctacagaagcaggcagtggctggcacgagatgggtctcctcgacttctcctacagcttcctgctcagagccggctacctgactctttacggaattgaggcgctgccacgcacccatgaaagccaggcccaggaccgcgtccactcagctgatgtcttccacacctttcgccagctcgaccggctgctccccaaactggcccgtggctccctgtcagtgggggacaaggaccacatgtgcagtgtcaaaagtcgcctgtggaagctgctatccccagccaggctggccaggcgggcccaccggagcaaatggctggagagttacctgctgcacctggaggagatgggtgtgtcagaggagatgcaggcacgggccctggtgctgcagctgtgggccacacaggggaatatgggtcccgctgccttctggctcctgctcttccttctcaagaatcctgaagccctggctgctgtccgcggagagctcgagagtatcctttggcaagcggagcagcctgtctcgcagacgaccactctcccacagaaggttctagacagcacacctgtgcttgatagcgtgctgagtgagagcctcaggcttacagctgcccccttcatcacccgcgaggttgtggtggacctggccatgcccatggcagacgggagagaattcaacctgcgacgtggtgaccgcctcctcctcttccccttcctgagcccccagagagacccagaaatctacacagacccagaggtatttaaatacaaccgattcctgaaccctgacggatcagagaagaaagacttttacaaggatgggaaacggctgaagaattacaacatgccctggggggcggggcacaatcactgcctggggaggagttatgcggtcaacagcatcaaacaatttgtgttccttgtgctggtgcacttggacttggagctgatcaacgcagatgtggagatccctgagtttgacctcagcaggtacggcttcggtctgatgcagccggaacacgacgtgcccgtccgctaccgcatccgcccatgacacagggagcagatggatccacgtgctcgcctctgcccagcctgccccagcctgccccagcctcccagctttctgtgtgcacagttggcccgggtgcaggtgctagcattaccacttccctgcttttctcccagaaggctgggtccaggggagggaaaagctaagagggtgaacaaagaaaagacattgaaagctctatggattatccactgcaaagttttctttccaaaatcaggctttgtctgctcccaattcacctcgttactctcacctcgtgatatccacaaatgctattcagataaggcagaactaggagtcttcactgctctgcccccaactcccggaggtgtcaccttcctagttcttatgagctagcatggcccgggccttatccagtcaaagcggatgctggccacagaaaggccactcaggatgtcctttgtgtcc

SEQ ID NO: 2 - protein, 500 aa, Homo sapiens
MAWAALLGLLAALLLLLLLSRRRTRRPGEPPLDLGSIPWLGYALDFGKDAASFLTRMKEKHGDIFTILVGGRYVTVLLDPHSYDAVVWEPRTRLDFHAYAIFLMERIFDVQLPHYSPSDEKARMKLTLLHRELQALTEAMYTNLHAVLLGDATEAGSGWHEMGLLDFSYSFLLRAGYLTLYGIEALPRTHESQAQDRVHSADVFHTFRQLDRLLPKLARGSLSVGDKDHMCSVKSRLWKLLSPARLARRAHRSKWLESYLLHLEEMGVSEEMQARALVLQLWATQGNMGPAAFWLLLFLLKNPEALAAVRGELESILWQAEQPVSQTTTLPQKVLDSTPVLDSVLSESLRLTAAPFITREVVVDLAMPMADGREFNLRRGDRLLLFPFLSPQRDPEIYTDPEVFKYNRFLNPDGSEKKDFYKDGKRLKNYNMPWGAGHNHCLGRSYAVNSIKQFVFLVLVHLDLELINADVEIPEFDLSRYGFGLMQPEHDVPVRYRIRP

SEQ ID NO: 3 - DNA, 20 bp, Artificial
  An artificially synthesized primer sequence
cagagagggtgggaatgacg

SEQ ID NO: 4 - DNA, 24 bp, Artificial
  An artificially synthesized primer sequence
gtgccaacaggattgaggagcgga

SEQ ID NO: 5 - DNA, 20 bp, Artificial
  An artificially synthesized primer sequence
gtcgggattttatgaatgaa

SEQ ID NO: 6 - DNA, 21 bp, Artificial
  An artificially synthesized primer sequence
gactgcagtcctggctcttgc

SEQ ID NO: 7 - DNA, 21 bp, Artificial
  An artificially synthesized primer sequence
tgagtccacagctgagatgcc

SEQ ID NO: 8 - DNA, 20 bp, Artificial
  An artificially synthesized primer sequence
gtgggccgctctaggcacca

SEQ ID NO: 9 - DNA, 25 bp, Artificial
  An artificially synthesized primer sequence
cggttggccttagggttcagggggg

SEQ ID NO: 10 - DNA, 23 bp, Artificial
  An artificially synthesized primer sequence
gggttgctgggggaagaaatgtg

SEQ ID NO: 11 - DNA, 23 bp, Artificial
  An artificially synthesized primer sequence
ggtggctgttttggtaggctgtg

SEQ ID NO: 12 - RNA, 21 nt, Artificial
  An artificially synthesized siRNA sequence
ggaaacggcugaagaauuauu

SEQ ID NO: 13 - RNA, 21 nt, Artificial
  An artificially synthesized siRNA sequence
uaauucuucagccguuuccuu

SEQ ID NO: 14 - RNA, 21 nt, Artificial
  An artificially synthesized siRNA sequence
gcggagagcucgagaguauuu

SEQ ID NO: 15 - RNA, 21 nt, Artificial
  An artificially synthesized siRNA sequence
auacucucgagcucuccgcuu

SEQ ID NO: 16 - RNA, 21 nt, Artificial
  An artificially synthesized siRNA sequence
ccggcuaccugacucuuuauu

SEQ ID NO: 17 - RNA, 21 nt, Artificial
  An artificially synthesized siRNA sequence
uaaagagucagguagccgguu

SEQ ID NO: 18 - RNA, 21 nt, Artificial
  An artificially synthesized siRNA sequence
ucaacagcaucaaacaauuuu

SEQ ID NO: 19 - RNA, 21 nt, Artificial
  An artificially synthesized siRNA sequence
aauuguuugaugcuguugauu

SEQ ID NO: 20 - RNA, 21 nt, Artificial
  An artificially synthesized siRNA sequence
uucuccgaacgugucacgunn

SEQ ID NO: 21 - RNA, 21 nt, Artificial
  An artificially synthesized siRNA sequence
acgugacacguucggagaann

SEQ ID NO: 22 - DNA, 154 bp, Homo sapiens
gactgtggtcacagccacagtgcactaagactatctgctcagcacttctggtgacccaaaagggtctgaggacaggagctcagagttgggtcagctgtccaggtactcagggttgtcacaggcaaaactgctggaactcagggcagcattgcaa